(19) United States
(12) Patent Application Publication
(10) Pub. No.: US 2025/0288223 A1
(43) Pub. Date: Sep. 18, 2025

(54) DEVICE AND METHOD FOR ADAPTIVE HEARING ASSESSMENT

(71) Applicant: JACOTI BV, Wevelgem (BE)

(72) Inventors: Kamil BUDZYNSKI, Barcelona (ES); Nun MENDEZ RODRIGUEZ, San Cristobal de la Laguna, Tenerife (ES); Amaury HAZAN, Barcelona (ES); Jacques KINSBERGEN, Antwerpen (BE)

(21) Appl. No.: 18/859,623

(22) PCT Filed: Apr. 28, 2023
(86) PCT No.: PCT/EP2023/061270

§ 371 Date: Oct. 24, 2024

Related U.S. Application Data
(60) Provisional application No. 63/336,015, filed on Apr. 28, 2022.

(51) Int. Cl. A61B 5/12; G16H 50/30

(52) U.S. Cl. CPC A61B 5/123; G16H 50/30

(57) ABSTRACT

A method for performing a hearing assessment procedure includes: performing a computational prediction of a hearing loss at at least one frequency different from one or more given frequencies for which a hearing loss indication is obtained by measurement, selecting a configuration for performing the hearing assessment procedure using at least the predicted hearing loss at the at least one frequency.

### FIELD OF THE INVENTION

The present invention is generally related to the field of devices and methods for conducting a hearing test, in particular a hearing test based on pure tone audiometry or a derivative thereof.

### BACKGROUND OF THE INVENTION

There are different methods to measure and describe a hearing loss. The characteristics of the methods are different for example in terms of accuracy, reliability and confidence levels. Methods can roughly be divided in two types, namely methods of testing which are accurate, have a high level of confidence and display a good test-retest reliability, and methods referred to as ‘predictive’, which allow for either measurement or prediction of a hearing loss with low accuracy (i.e., deviating from the clinical audiometry gold standard), low level of confidence (i.e., confidence of prediction) and low test-retest reliability. Some of these predictive methods do not predict the hearing loss, but rather select hearing loss compensation parameters based on user preference.

Pure tone audiometry is considered the gold standard among the hearing tests. It is used to identify hearing threshold levels of an individual person, enabling a determination of the degree of hearing loss at different frequencies. Pure tone audiometry is a subjective measurement of a hearing threshold (an audibility threshold), as it relies on the individual's responses to pure tone stimuli. Pure tone audiometry provides ear specific thresholds, and uses frequency specific pure tones to give place specific responses, so that the configuration of a hearing loss can be identified. Calibrated audiometry headphones are typically used, sounds are presented by using a pure tone audiometer whereas a tone is presented by the test leader and the test person is expected to respond to this by pushing a button, raising the hand, giving a voice signal or, for children, by performing an action in a play situation. A quality of the assessed pure tone thresholds, i.e. the lowest level heard by the patient, not only depends on the reliability of the test person's responses, but also very strongly on the competencies and experience of the test leader.

In EP2572640 B1, incorporated in the present document by reference, another example of hearing assessment based on accurate measurements is presented. The patent discloses a method (further also referred to as the ‘DuoTone’ method) for conducting a pure tone audiometry, wherein tones of different frequency and intensity are utilized in an adaptive procedure. The tone signals with at least two different frequencies are independently changed by delivering to a test person various test stimuli. Each test stimulus is randomly selected from a set of at least three different test stimuli. The different test stimuli comprise either no tone at all, one tone with a first frequency or a multitude of short tones with a second frequency higher than the first frequency. Various studies have indicated this method offers a similar accuracy to the Pure Tone Audiometry approach and also shows high test-retest reliability. DuoTone allows measuring hearing thresholds with high confidence levels.

The test procedure for each individual frequency works as follows: once the test person has prompted a correct answer to a test stimulus with a set frequency, the next test stimulus at that frequency is presented with a lower intensity, typically with a step size of 10 dB. Other step sizes, however, can obviously also be used. If the test person has given a wrong answer, the next stimulus presented to the test person with that frequency is higher again, usually twice the normal step size. This procedure is usually repeated three times so that three lower hearing thresholds are determined for each frequency utilized in the test procedure.

In that way the lowest intensity, just heard by the test person, can be reliably determined. Once the measurement system has found the lowest intensity for the same frequency for the third time, the method is ended for that frequency and the result, i.e. the threshold value, is determined.

In the current implementation of DuoTone, only two distinct configurations are being used:
a short test, wherein four frequencies are tested in one session
a long test, wherein twelve frequencies are tested in one session
   For the short hearing test the four frequencies tested are 500 Hz, 2 kHz, 1 kHz and 4 kHz. For the long hearing test the used frequencies are 500 Hz, 2 kHz, 1 kHz, 4 kHz, 125 Hz, 8 kHz, 250 Hz, 12 kHz, 750 Hz, 3 kHz, 1.5 kHz, 6 kHz. Each individual frequency is tested with the same accuracy.

An important characteristic of hearing assessment methods to be considered is the duration of the hearing test. Especially in case of a self-test this is an important factor for many users. A DuoTone long test procedure testing 12 frequencies and optimised for high accuracy, takes around 10 minutes. This is already a substantial improvement over a Pure Tone Audiometry, which can take 20 to 30 minutes.

In the current implementation of DuoTone, the algorithm always starts testing a hearing threshold at a constant level, e.g. 50 dB HL. This is so regardless of any information about the subject that might be available. Each test session thus starts without knowledge about the user.

However, the DuoTone hearing test maintains its accuracy regardless of the starting point. If a user's hearing is better than the starting point value, the DuoTone algorithm continues decreasing the intensity, until a threshold is reached. In case of the user's hearing being worse than the starting point, the algorithm increases the intensity and determines hearing loss levels accordingly.

The DuoTone hearing test continues generating stimuli until an exact hearing threshold can be calculated or the hearing threshold is outside of supported range. The latter can happen in two cases:
the hearing loss is too high, therefore it is defined as a range [X, infinity], where X is the maximum supported value
the hearing loss is too low (either due to ambient noise, hardware limitations or design choice), therefore it is defined as a range [-infinity, X], with X the minimum supported value

From the above it is clear there are various improvements possible to the DuoTone approach as currently used in the art. The DuoTone approach is however only one possible procedure for performing hearing assessment. By extension, also for other hearing assessment procedures improvements are possible.

### SUMMARY OF THE INVENTION

It is an object of embodiments of the present invention to provide for a rapid method for hearing assessment. It is a further object of the invention to provide for a system for performing such a method.

The above objective is accomplished by the solution according to the present invention.

In a first aspect the invention relates to a method for performing a hearing assessment procedure comprising
performing a computational prediction of a hearing loss at at least one frequency different from one or more given frequencies for which a hearing loss indication is obtained by measurement,
selecting a configuration for performing the hearing assessment procedure using at least the predicted hearing loss at said at least one frequency.

The proposed solution indeed allows for obtaining a scheme for a hearing assessment procedure that can be performed in a fast way. Due to the predicted hearing loss one has a starting value for one or more frequencies that are used in the hearing assessment procedure. The proposed method further offers the advantage of easily allowing for an optimisation to specific needs without sacrificing accuracy. For example, the number of frequencies used in the procedure directly relates to the time required for performing the hearing assessment. This holds even more if, as in preferred embodiments of the invention, additional parameters are taken into account to perform the prediction and select the configuration.

In a preferred embodiment the computational prediction is based on at least one of {noise analysis, keyword detection in speech, dynamics of hearing loss changes over time}. Monitoring and analysis of noise present while performing the test procedure may yield additional information that allows finetuning the accuracy of the determined thresholds. Regular use of utterances like for example ‘Can you repeat?’ in a conversation may be a pointer of a hearing problem. Hence, detection of often used words in such phrases can provide useful information. The dynamics of the hearing loss can for example account for an observed hearing deterioration rate over a certain amount of time. Possibly this may involve observing changes at various frequencies.

In some embodiments the computational prediction is based on a volume setting and/or gain setting in a device used to provide test stimuli during the hearing assessment procedure.

Advantageously the computational prediction may further be based on at least one of {demographic information, situation analysis, hearing loss indications determined in the past, information on a configuration used in the past, hearing loss at frequencies different from the frequencies being tested in the present hearing assessment procedure}.

In some embodiments a weighting is applied of two or more pieces of input information when performing the computational prediction.

In a preferred embodiment each stimulus used in said hearing assessment procedure is randomly selected from a set of at least three different testing signals comprising no tone at all, one tone with a first frequency or a plurality of tones with a second frequency different from said first frequency.

In preferred embodiments the computational prediction of the hearing loss is expressed as a range.

In one embodiment during the hearing assessment procedure additional information for the prediction is supplied and an update of the configuration is determined based on an updated hearing loss prediction.

Advantageously, the hearing assessment procedure is performed both at at least one frequency for which a prediction is computed and at at least one frequency of the one or more given frequencies.

In a preferred embodiment the configuration comprises a starting point for the procedure and a step size to adapt the intensity of stimulus between successive stimuli used in the procedure.

In another embodiment the method comprises a step of determining a correlation between inputs.

The method as set out above is preferably computer-implemented. In one aspect the invention relates to a program, executable on a programmable device containing instructions which, when executed, perform the method as previously described.

In one aspect the invention relates to a system for performing a hearing assessment, comprising a selection module arranged for receiving parameter values and for selecting a configuration for performing the hearing assessment, processing means for performing a computational prediction of a hearing loss at at least one frequency different from one or more given frequencies for which a hearing loss indication is obtained by measurement, and tone generation means for generating tones according to the selected configuration.

In some embodiments the selected module and/or the processing means and/or the tone generation means are integrated.

In embodiments the selected module and/or the processing means are distributed over at least two physically separated devices.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

The above and other aspects of the invention will be apparent from and elucidated with reference to the embodiment(s) described hereinafter.

### BRIEF DESCRIPTION OF THE DRAWINGS

The invention will now be described further, by way of example, with reference to the accompanying drawings, wherein like reference numerals refer to like elements in the various figures.

FIG. 1 illustrates the building of a training data set for the prediction model.

FIG. 2 illustrates a selection module of a system for conducting audiometry screening and its possible inputs for making a hearing loss prediction.

FIG. 3 illustrates a selection module distributed between two devices of the system.

FIG. 4 illustrates a selection module distributed between three devices.

FIG. 5 illustrates a selection module distributed between two devices (a server and a mobile device), where a third device executes the hearing test.

### DETAILED DESCRIPTION OF ILLUSTRATIVE EMBODIMENTS

The present invention will be described with respect to particular embodiments and with reference to certain drawings but the invention is not limited thereto but only by the claims.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

It should be noted that the use of particular terminology when describing certain features or aspects of the invention should not be taken to imply that the terminology is being re-defined herein to be restricted to include any specific characteristics of the features or aspects of the invention with which that terminology is associated.

In the description provided herein, numerous specific details are set forth. However, it is understood that embodiments of the invention may be practiced without these specific details. In other instances, well-known methods, structures and techniques have not been shown in detail in order not to obscure an understanding of this description.

The present invention aims to propose a device/system and method to perform hearing assessment in an improved way, whereby results with high accuracy, high confidence level and good test-retest reliability are obtained in a faster way than with the prior art approaches.

In the proposed solution first a computational prediction is performed of the hearing loss based on inputs taken from a wide variety of possible inputs as described below. An adapted/tailor made configuration for the hearing assessment procedure, e.g., a DuoTone procedure as described above, is then determined using the outcome of the prediction stage. When performing the hearing assessment using the configuration as determined a correction and validation of the prediction stage is carried out in order to ensure accuracy and reliability.

In a minimum set-up a prediction of the hearing loss at at least one additional frequency is made based on the hearing loss at at least one given other frequency obtained via measurement. In this way starting points for assessing hearing loss are obtained for a set (superset) of frequencies larger than the subset for which measurements are already available. Additionally, the prediction may be based on other input data as described below.

The prediction model allowing estimation of the threshold for a superset frequency based on a subset frequency threshold measurement may be built in the following way. A database containing audiograms in which the thresholds for the subset frequencies have been measured, is used to start with. An audiogram is a representation of a person's hearing compared to the average young normal hearing person. A training data set is built: each row in the database comprises input and target columns. Input columns contain thresholds for all subset frequencies, and target columns contain the thresholds for all superset frequencies. Superset frequencies may either be frequencies present in the subset or may lie between two subset frequencies. In the latter case, thresholds for superset frequencies can be computed as an interpolation, e.g. a linear interpolation, of known thresholds at subset frequencies. Once the training data set is built, a prediction tool, i.e. a multidimensional regression model, is trained to predict superset frequency thresholds based on subset frequency thresholds, for each row of the data set. For each row, predicted superset thresholds are compared with actual thresholds for the superset frequencies using a multidimensional regression error metric, such as a root mean square error. Training is performed over the training data set to minimize said multidimensional regression error metric, with the effect of adjusting the parameters of the multidimensional regression model. Therefore, a trained model comprises a multidimensional regression model in which the parameters have been adjusted to minimize the error between predicted and actual superset frequency thresholds over the training data set. Examples of multidimensional regression model that can be trained using the method outlined above include but are not limited to: linear regression models, artificial neural networks, bayes networks, decision trees, support vector machines.

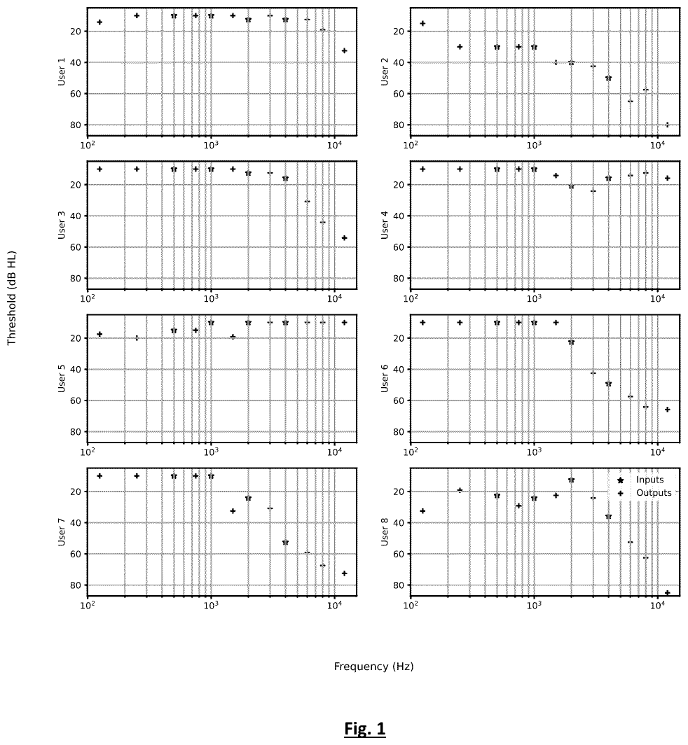

FIG. 1 illustrates how the training data set is built. FIG. 1 shows entries of the database that are used to build a dataset in order to train a prediction model of hearing thresholds at superset frequencies (marked with ‘+’ as outputs) based on thresholds at subset frequencies (marked with ‘*’ as inputs). In this example measured threshold levels at frequencies of 500, 1000, 2000 and 4000 Hz (forming the subset) were used. The superset of frequencies comprises apart from the threshold levels of the subset frequencies also threshold levels obtained by prediction at 125, 250, 750, 1500, 3000, 6000, 8000 and 12000 Hz.

The prediction model is stored in a memory. In some embodiments the selection module as further described below is provided with storage means where the prediction model can be stored. In other embodiments the prediction model may be stored in memory of the consumer device or in the cloud.

In embodiments of the invention, one or more inputs may be gathered in the prediction model in order to compute a prediction of hearing thresholds as accurate as possible. Some of the inputs may be applied to the model using a user interface, for example a graphical user interface. The accurate predictions may use statistical methods and possibly machine learning techniques to build a prediction of hearing thresholds using information described in detail below. By combining data inputs like for example one or more of user behaviour, historic test results, user input and statistical data of thousands of users, without being limited thereto, it is possible to create computational predictions with predetermined confidence levels. The predetermined confidence levels may for example be 90% within +/−5 dB HL. This is in contrast to traditional approaches, where this amount and type of data was not used when predicting hearing loss. The predictions are next validated. Conventionally, accurate hearing assessment methods are used in a fixed configuration, without consideration of information already available or, for example, of statistical information about similar persons. This ensures repeatability of the test, but at the same time does not allow for optimisations. Such a situation results in long testing times as already mentioned above.

In preferred embodiments prediction thresholds are computed considering also information derived from noise analysis. Noise is monitored for example while the test is being performed. This info may in some embodiments be fed back to the selection module so that the configuration can be changed on-the-fly if needed. Further also information related to long term exposure to noise can provide useful additional input. Such information may be stored e.g. in a database accessible for the selection module. Also detection of spoken keywords can advantageously be used as an indicator of potential hearing loss. People who regularly use sentences like “I can't hear”, “can you repeat” may have hearing difficulties. Such phrases can be detected by means of a microphone comprised in the device or system and processed with conventional speech recognition means. Another useful parameter may be the observation of dynamic changes in hearing loss. For example, if the hearing loss threshold at one or more test frequencies is found to deteriorate with 5 dB per year is found for a given person, this information can be used when determining starting values for performing a hearing assessment on that person. Further, a situation analysis can be carried out to describe the (environmental) context wherein the hearing assessment takes place. Such an analysis may lead to attributing one of a set of predetermined acoustic scene categories to the current situation. For example, different acoustic scene categories may have been identified upfront based on the total amount of environmental noise, noise frequency characteristics, directionality and dynamics of changes in noise. An example of such categories may be: “silent room”, “silent room with low frequency noise”, “street with high level of traffic”.

Auditory masking effect is a known process of masking sounds by the presence of different sound (e.g., environmental noise). The presence of background noise can mask or interfere with the perception of pure tones, leading to inaccurate hearing test results. By classifying the environment in terms of acoustic characteristics, one can predict the auditory masking effect during the hearing test. In order to achieve higher accuracy, it is possible to select configuration of the hearing test (e.g., selection of frequencies) that will be the least affected by auditory masking in the current acoustic environment.

In an advantageous embodiment also information on the volume setting and/or microphone gain setting in the consumer device used as input device for the test stimuli is exploited for computational prediction. The consumer device may for example be a pair of earbuds, a headphone, a smartphone or a tablet, without being limited thereto. Although the volume setting does not depend on frequency, it gives a general indication of the experienced hearing loss: a user who suffers from more hearing loss, tends to increase the unfitted volume setting of the device. The information about volume setting and its variability is available to the logic controlling the consumer device's operation and is advantageously taken into account in the selection module when making a hearing loss prediction. It allows determining a more accurate starting point for one or more of the various frequencies considered in the test procedure. Loudness of user speech can also be used as an indicator of potential hearing loss. People with a hearing loss tend to speak louder than people with normal hearing. So, behavioural patterns like volume settings, changes of volume over time, speech loudness may provide relevant additional information for selecting a configuration for the hearing assessment procedure.

Further deviating from the conventional approach, as adopted for example in the DuoTone procedure wherein always the same starting point is used, one can also take into account one or more of the following pieces of additional information in order to obtain a computational prediction and configuration:
previously determined or predicted hearing loss, possibly at frequencies different from frequencies considered in the current test procedure
demographic characteristics
previous testing session details (whether some frequencies were successfully tested, amount of noise, number of errors, etc.)
acoustic scene categorization obtained from a situation analysis
sensor data (e.g. environmental, heart rate, EEC, ABC (altimeter, barometer and compass), IMU data (Inertial Measurement Unit))
context aware information (time of the day, geographical location, three-dimensional position) standardised questionnaire (e.g., questions about tinnitus, hearing difficulties, behaviour in crowded places)
personalised expert input (e.g., frequencies selected by audiologist, personalized questionnaire)
external input parameters, such as
   Test length and priority
   Desired accuracy
   Use case
   Hardware device characteristics (ear seal in lower frequencies, passive and active attenuation)
   Desired number of frequencies tested in one session
   User preferences
      Note that this list is non-exhaustive. Some of the possible inputs are illustrated in FIG. 1.
      This information can be exploited not only when computing the prediction but also when taking a decision on the order wherein frequencies are tested, i.e. when selecting a configuration.

A weighting of parameters considered when carrying out the method of the invention comprising computing the prediction and selecting a configuration, can be performed by means of conventional techniques known in the art, e.g., linear regression. Weights can be expressed as constant values or as a function of one or more parameters, e.g., time or location. This allows the weights to change dynamically, thereby also changing the decision process. The weights in this approach may also have preset values for a given particular use case. The use case is inputted to the control logic of the selection module and may be provided by user choice, location, AI methods or other.

By using information from this multitude of possible inputs, one can obtain hearing loss predictions with medium to high accuracy and confidence level (e.g., 70% within +/−20 dB HL, 90% within +/−5 dB HL). The computational prediction may in some embodiments be based on one or more of the above-mentioned parameters, like for example at least one frequency for which hearing loss information is already available by measurement and one frequency for which a hearing threshold prediction has been made, volume settings of the device used for performing a test procedure and, optionally, past results, demographics and/or behavioural patterns. By itself this prediction may already have a high accuracy in some cases (e.g., if one has access to the recent hearing test history), but in order to ensure accuracy and reliability, a correction and validation step is necessary.

The computational prediction output data is next used to select a configuration for a hearing assessment procedure, e.g. a pure tone audiometry or a DuoTone method. The output data may comprise a list of frequencies to be used when testing, hearing thresholds and confidence levels. Based on at least the computational prediction output (e.g., hearing thresholds and confidence levels) one can configure an optimised assessment procedure aiming at the validation of hearing thresholds rather than discovery of mentioned thresholds. When hearing thresholds with high confidence level are available, the same final accuracy can be obtained with fewer steps. The assessment procedure can focus on the hearing region where a hearing threshold should be found, with a fallback mechanism provided in case the prediction should be less accurate. Such usage of hearing thresholds is not applied in traditional approaches, due to low accuracy, lack of availability of the data and hearing assessment being manually performed by audiologists.

The skilled person will readily understand that the above-mentioned pure tone audiometry and a DuoTone method are only two (advantageous) examples of possible hearing assessment procedure that can be applied with the proposed invention. The invention can also be applied to other audiometric tests, including, but not limited to:
Sound field audiometry, in which stimulations are produced with a speaker instead of using earphones
Audiometric tests involving stimulation different than pure tone, e.g. warble tones
Visual reinforcement audiometry and play audiometry, targeted at children
More generally, any audiometric test in which a hearing assessment metric is given over a plurality of frequencies in order to represent a measure of hearing ability over the frequency spectrum

In preferred embodiments a method of configuring a DuoTone test procedure is presented, which is optimised for a number of parameters-for example, test duration, expected user fatigue, use case, accuracy. As mentioned in the background section, in the past this method was executed in a repeatable manner, without considering additional information. Having a multitude of pieces of information available, with a computational prediction, it is possible to create custom configurations, which can be optimised according to specific needs. Due to the robust nature of a DuoTone test, the hearing assessment result is highly accurate.

In an approach like DuoTone, for example, the time to obtain results of a hearing test is directly proportional to the number of frequencies being tested. The time to determine a hearing threshold value for one frequency is directly proportional to the distance between the hearing loss level taken as starting point and actual hearing threshold for that frequency. It is also directly proportional to the step size when lowering intensity of the tone and inversely proportional to the step size when increasing the intensity (assuming the starting point is higher than the hearing threshold). Further optimizations of the correction and validation step can be done by using information as described above.

In one embodiment the hearing assessment procedure, e.g., the DuoTone test, can be configured in a way that is optimised for total test duration. Scheduled is a single test session, that consists of four frequencies being tested. The selection of frequencies focuses on the use case (telephony) and frequencies around speech were selected. This allows having an accurate representation of hearing in the speech region within three minutes.

In another embodiment the hearing assessment procedure is configured with emphasis on accuracy and potential user fatigue. Scheduled are two (or more) test sessions on different days. Each session may consist for example of four frequencies. In order to avoid user fatigue, a big step size is used in the beginning, which is lowered when approaching the actual hearing threshold for a given frequency. The result of the test performed in the various sessions is an audiogram consisting of eight frequencies. Validation can be performed within two sessions of two minutes each.

In one aspect the invention relates to a device or system for performing the method as described herein. A key component of such a device or system is a selection module, also referred to in this description as selection block. The module is typically implemented in software. In one embodiment, however, the selection module can be realized in hardware. This module decides, based on the available hearing thresholds (either measured or calculated in the prediction step) and possibly on other input parameters, on how the hearing assessment procedure is carried out, i.e. which configuration is applied (for example, which frequencies are used, which step size etc). The selection module selects the parameter values to configure the procedure. In one embodiment the configuration is selected from a set of predetermined configurations stored in a memory, for example a memory of a processing device interacting with the selection module or storage means provided in the selection module itself. In other embodiments the configuration is calculated on the fly, using the available input information. In some embodiments the selection module is also capable of computing hearing thresholds using the prediction model. In other embodiments this computation of thresholds based on the prediction model is performed in a processing device in connection with the selection module. The control logic in the selection block is in some embodiments arranged to receive various types of input. In embodiments where the threshold computation is performed in a separate processing device, that processing device is arranged to receive the input information. The input information can then be used in the determination of the predicted hearing loss and next in the configuration of the hearing assessment.

In this description control logic, as comprised among other things in the selection module, refers to a set of software instructions to control the device or system and the application it is part of. It controls a sequence of operations to be executed by the computational and memory components of the device or system. Control logic may react in response to commands received from a user, but it can act also on its own to perform certain tasks. The control logic in the selection module receives, optionally monitors, a set of parameters and takes a decision on which actual configuration to use for the hearing assessment to be performed so that the execution of the hearing assessment algorithm is optimized in terms of the given requirements for a given use case. Additionally, the control logic in the module may change the way the decision making process is performed, for example by adjusting one or more other parameters used when taking the decision. The monitoring and optionally adjusting can in some embodiments be performed for example at fixed, possibly regular, time intervals. In other embodiments continuous or quasi-continuous monitoring and adjusting can be applied.

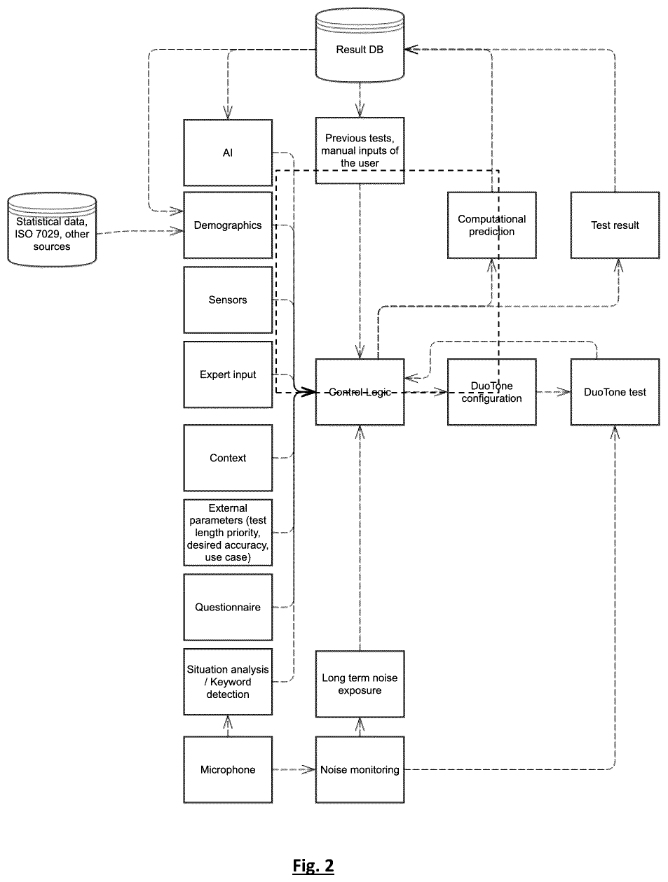

Apart from an overview of the possible inputs to the selection module, FIG. 2 also illustrates some possible implementations. In one embodiment the selection module corresponds to the rectangle in dashed line in FIG. 2. In the embodiment depicted in FIG. 2 a DuoTone configuration is determined in the selection module. The selection module outputs, based on at least some of the parameters shown in FIG. 2, a configuration for performing a hearing assessment procedure according to the invention to evaluate and correct the hearing loss values. The solution can thus be seen as an improved DuoTone procedure. The configuration may comprise information on the number of test sessions and how to schedule them, on the frequencies selected for each of the test sessions and the configuration to be used for each frequency (starting values for the hearing loss, a step size, which may possible be variable, and a stop condition). It is repeated that other implementation than shown in FIG. 2 are possible. For example, as already mentioned previously, in other embodiments the computational prediction may be calculated external to the selection module.

The selection module is in preferred embodiments part of a consumer electronics device, like e.g. a personal computer, laptop, tablet, smartphone, smartwatch, an ear-level processing device or another portable computing device. The term ear level audio processing device is used in this description to refer to any device that, when in use, resides at ear-level and comprises at least one audio output and some means for standalone processing (e.g. a DSP and/or a CPU). The device is further in this description sometimes called ear-level processing device or ear-level computing device. Some examples of an ear-level processing device are a Bluetooth headset or a so-called smart headset. Smart headsets are technically advanced, electronic in-the-ear devices designed for multiple purposes ranging from wireless transmission to communication objectives, medical monitoring and so on. Smart headsets combine major assets of wearable technology with the basic principles of audio-based information services, conventional rendition of music and wireless telecommunication.

The consumer hardware device is provided with conventional components like processing means (e.g. a digital signal processor (DSP) and/or a central processing unit (CPU), possibly a multi-core CPU), and optional memory. In some embodiments the processing means is coupled to a digital-to-analog converter and output transducers to emit the generated test stimuli. The tone generation delivers pure tones of different frequencies and intensities. One or more processing means is available with which the selection module can cooperate. In some embodiments the processing means are an integrated part of the hardware device. In other embodiments the processing means may be non-integrated, i.e., physically separated from the consumer hardware device. In this case one rather obtains a system comprising at least one hardware device and one or more processing means. The selection module may be part of a processing means in some embodiments. In other embodiments the selection module, and consequently also the control logic comprised in the selection module, may be distributed over a number of processing means. In that case, the processing means may be in connection with each other so that communication between them is possible. The connection may for example be a wireless link, e.g., a Bluetooth communication link, or a wired link.

In some embodiments the consumer hardware device may be acoustically calibrated. Acoustic calibration involves a characterisation of selected transducers or all transducers available on the device (microphones and speakers). Characterisation may involve measuring relation between digital and acoustic signal of transducer (sensitivity, linearity), measured in, e.g., third octave bands.

In some embodiments the selection block is arranged to process in the first step available data to perform the computational prediction. In other embodiments the prediction may be computed in a processing means of the device or system external to the selection module and fed to the module. Next, in a second step, the control logic in the selection module generates a hearing assessment configuration. In optional later steps the control logic may perform validation and auto-optimisation of parameters when new information becomes available during the session. At each moment, the control logic in the selection module can output information about hearing thresholds, associated confidence levels and additional information (e.g., tinnitus characterisation, diagnosis, probability of hearing loss, hearing age).

Based on the selected parameters a prediction of the hearing loss is computed. In some embodiments the control logic in the selection module thereby provides, at least for the frequencies selected for carrying out the test, predicted hearing thresholds and corresponding confidence levels. The hearing thresholds may in other embodiments be input into the selection module after having been computed in a processing means in the hardware device or even external to that device. Further the selection module provides a test schedule and configuration for verification of the predicted hearing loss levels. The frequencies selected for use in the test session are indicated as well as the starting point, the step size (or step sizes) to be applied and a stop condition.

A specific example of a possible resulting procedure provided by the control logic of the selection module may be a type of prescreening, wherein users that may have a hearing loss, are filtered out from those whose hearing is most probably normal. A high-level requirement for the length of such a prescreening phase is about 30 seconds, i.e., it should not last longer than 30 seconds. The selection module may provide a set of frequencies optimized in number and frequency values, an optimized step size and an optimized starting point. All these values may be obtained by properly weighting input parameters (use case, prior knowledge about the user, accuracy, binary result type, duration). A resulting test configuration may for example consist of two frequencies, a variable (and initially big, for example 20 or 30 dB) step size, a low starting point and a stop condition adjusted for normal hearing. For example, a first test is started using tones at 500 Hz and 4 kHz. In case the predicted thresholds at both frequencies are found satisfactory, the procedure stops. In case the threshold at one or both frequencies is not adequate, a subsequent test procedure using frequencies of 1 kHz and 2 kHz is started.

As previously explained the hearing loss threshold levels are adapted with one step of a given size at the time. If there is a major difference between the starting point and the actual hearing threshold (positive or negative), many iterations and, hence, a lot of time will be needed to reach the threshold level. Therefore, it is advantageous to have a good estimate available of the threshold level at a given frequency for a given user and to use that value as a starting point when performing the procedure. A constant offset from the threshold level may be taken into account. In that way testing of as few frequencies as possible may lead to the shortest procedure, as many steps can be avoided. As in theory one frequency might be sufficient, the proposed approach relies on calculating the hearing loss at at least one frequency for which no a priori hearing loss information is available. Note that in an approach as in DuoTone, at least two frequencies are used. The calculation is performed in the computational prediction step of the proposed method. The calculated starting point for assessing hearing loss, i.e., the calculated threshold levels at the selected frequencies, is then used in the configuration of the hearing assessment procedure to be carried out, possibly along with threshold levels that were obtained by measurement.

Another useful piece of information may be a previous result of a hearing test. Having data of previous hearing test results, the last obtained hearing threshold for each of the different frequencies can be used directly as a starting point. Assuming no fast deterioration of the hearing capability, this may give a good first estimation. In an alternative approach probable thresholds can be predicted by looking at a series of previous hearing tests and a hearing deterioration rate. Dynamic changes in the hearing loss can so become apparent.

In many cases one has access to some demographic information about the user, in particular about the age. A statistical model of the hearing loss can be built and used as starting point for accurate testing, for example in a DuoTone set-up. For users whose hearing is similar to the statistical model, the test can be performed faster. For other users with a deviating hearing profile, test potentially may take longer. The ISO 7029 standard is one example of a statistical model that can be applied. The standard provides descriptive statistics of the hearing threshold deviation for populations of otologically normal persons of various ages under monaural carphone listening conditions. It specifies for populations within the age limits from 18 years to 80 years for the range of audiometric frequencies from 125 Hz to 8 000 Hz, a) the expected median value of hearing thresholds given relative to the median hearing threshold at the age of 18 years, and b) the expected statistical distribution above and below the median value.

In one embodiment a machine learning model can be trained using an extensive database of user data. This may allow finding a correlation between different types of information (demographics, test history, software and device usage) and the computational prediction and configuration. Frequencies to be used in the test session can be selected taking into account, for example one or more of the following (without being limited thereto):
Session time constraints
Minimum and maximum number of frequencies
Demographic characteristics
Previous results (determined hearing loss, number of sessions, selection of frequencies, confidence levels, noise levels)
How old are results and probability of hearing deterioration
Progress of hearing loss over time
Tinnitus presence and frequency characteristic
   The model can be trained on a huge database of users (e.g. more than ten thousand) and multiple sessions. The database may comprise previous hearing assessment tests, including test details (e.g., error rate) and other information about users. Parameters mentioned above can be found helpful in determining the best configuration.

Another important parameter that affects the duration and accuracy of the hearing assessment procedure is the step size applied to increase or decrease the intensity of the tone being tested compared to the tone previously used in the test procedure. Various options are available.

In some embodiments the Hughson-Westlake procedure may be adopted, which proposes a method to search for hearing threshold by using the following rules:
when a user hears the tone, the intensity is decreased by 10 dB
when the user does not hear the tone, the intensity is increased by 5 dB
the lowest intensity is sought at which the user hears the tone at least 50% of the time
   In a procedure like e.g. DuoTone a similar approach can be employed (half the step size going up, instead of doubled step size as is currently used). Each reversal should result in a lower number of required steps to determine the hearing threshold.

Apart from determining a ratio between the applied step sizes when increasing or decreasing the intensity of a tone, also the absolute value of the step size can be variable. This can be selected for example based on following parameters:
Frequency tested
Number of reversals in a given frequency
Confidence levels
Priority of test length
Error rate in previous tests
   The selection block is adapted to adjust the parameters in real time, i.e., the parameters can be auto-optimized during the test.

Another way to optimize, i.e. to reduce, the time required to terminate a hearing test session is to select only a limited number of frequencies. This can be done in various ways depending on the situation at hand. For specific use cases some frequencies may not be needed, e.g. telephony is limited to 8 kHz. Hence, in such case a hearing can be tested for frequencies up to for example 6 kHz or 8 kHz, instead of going up to full 12 kHz. Other use cases can be defined and suitable frequencies selected.

If hearing test results at some frequencies are available, one can focus on testing different frequencies when performing a new hearing test. In other words, the frequencies for which a first indication of the hearing loss has been derived by interpolation as previously explained, are considered in the first place. The confidence level may be influenced by e.g., which hardware and/or software were used to determine the threshold, how old are the available results, which noise levels are present). If for example a part of the hearing thresholds already has high confidence levels, the hearing assessment correction and validation may focus on frequencies with a lower confidence level first.

Instead of testing e.g. 12 frequencies at once, the test can be divided into n (e.g., n=2) separate sessions (with the tests being performed e.g. on a different day) to prevent any effect of user's fatigue and to improve so the user experience. For example, the most important frequencies can be selected for the first test, and more fine-grained information can be obtained in a second session. Identifying the most important frequencies may be based on:
probable hearing loss (e.g., age related hearing loss)
target device/use case (e.g., test telephony first, music later)
   Some frequencies are more prone to hearing losses (e.g., age-related hearing loss), therefore higher resolution of testing in that region might be beneficial. In the proposed approach n sessions can be scheduled spread over time to optimise for the best user experience.

In some embodiments next frequencies to be used for testing can be determined by interpolation and extrapolation dependent on the hearing loss degree for frequencies already addressed in the test. This can be realized in the same way as was explained with respect to the frequencies to start the procedure with. For example, a first test is started using the frequencies of 500 Hz and 4 kHz. In case the predicted thresholds at both frequencies are adequate, the procedure stops. If not, a subsequent test procedure using frequencies of, for example, 1 kHz and 2 kHz is started. The interpolated frequencies selected to be tested next can be determined depending on factors like confidence level, age of user, etc.

Ambient noise can limit the intensity levels that can be tested at a given moment. The selection block selects in some embodiments frequencies where noise levels are sufficiently low, i.e. below a predefined threshold level, instead of using testing frequencies in which ambient noise is high.

By detecting situation and behavioural patterns it is possible to computationally predict the hearing loss and derive the best configuration of hearing test (including duration, selection of frequencies, etc.). This can be achieved by observing patterns (e.g., increased volume settings in past months, louder speech, keyword detection, head movement indicating hearing difficulties, error rate in previous tests) as described above. By detecting specific patterns it is possible to predict a degree of hearing loss. This information can be used to emphasize accuracy and test a higher number of frequencies, starting at higher level of hearing loss.

When the hearing loss level is low (close to normal hearing), approaches like the conventional DuoTone algorithm may not be able to accurately measure the hearing threshold. In that case, the algorithm specifies the hearing loss as a range, e.g., “20 dB hearing loss or better”. This means the user may have hearing loss of 20 dB, but he may have much better hearing as well. This information is sufficient as a result of hearing test (as the user's hearing is confirmed to be normal) but it may be insufficient to be used as an input to fitting a hearing aid. In the past, the result “20 dB or better” might have been used for fitting in two ways:
Provide gains appropriate for 20 dB hearing loss (HL)-this may cause overstimulation in some frequencies with no hearing loss. It will also provide less contrast between regions with hearing loss (e.g. 30 dB HL) and no hearing loss (“20 dB or better”)
Provide no gain, corresponding to no hearing loss, which may provide insufficient amplification in the region
   Using the approach presented in this invention a fitting is estimated for a user with hearing loss in form of a range. The calculation can take into account
Calculating an average of the hearing loss range (e.g. provide fitting for 10 dB HL for range [-infinity, 20])
Demographics (probability of age-related hearing loss and amplification needs)
Behaviour during the test (number of errors, how long it took to answer)

As already discussed above, in one aspect of the invention a device for performing the hearing assessment is proposed. In another aspect of the invention a system comprising a plurality of physical entities is proposed for performing the hearing assessment. This system or device comprises in some embodiments an integrated selection module and further contains at minimum:
Acoustic, digital or analog input and output (e.g. microphone, line-in, speaker, line-out)
Processing means (e.g. a processing unit like a CPU or DSP)
Memory and storage (RAM, flash memory)
User interface or wireless communication capabilities
   An example of such a device may be a pair of headphones, with integrated microphones, processing unit and custom user interface to perform the test.

Some further implementation aspects of the proposed device or system for conducting audiometry screening are discussed. As already mentioned, a key component in the system is the selection module. The selection module may in some embodiments be a part of the control logic block described in U.S. Pat. No. 9,055,377, which is hereby incorporated by reference. In other embodiments the selection module may be separate from the control logic block of U.S. Pat. No. 9,055,377.

The control logic in the selection module can be distributed between two and more devices or can be run on one device (e.g. mobile, server, ear-level device, other). Different tasks of control logic can be executed on any of the distributed parts of the control logic. Different inputs can be processed in any part of the control logic.

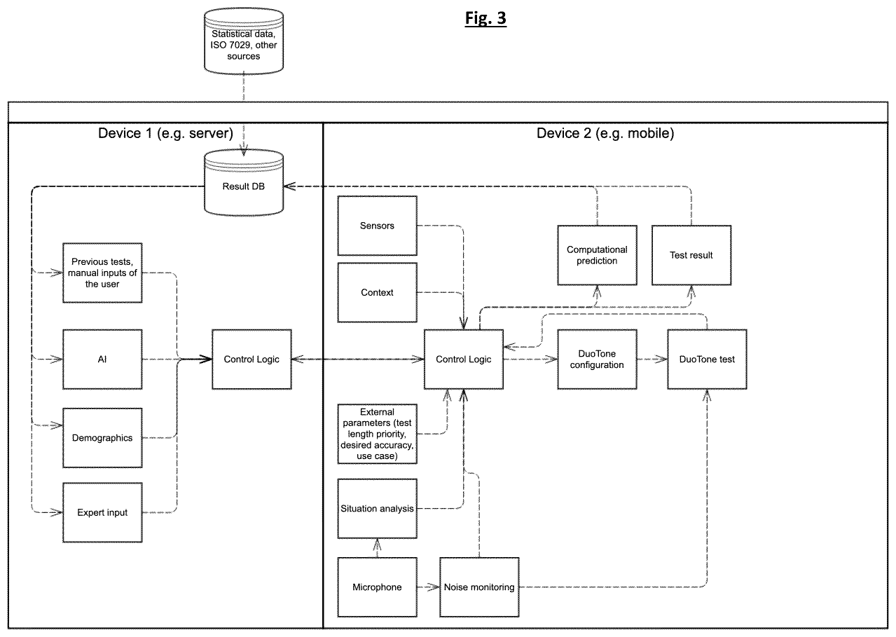

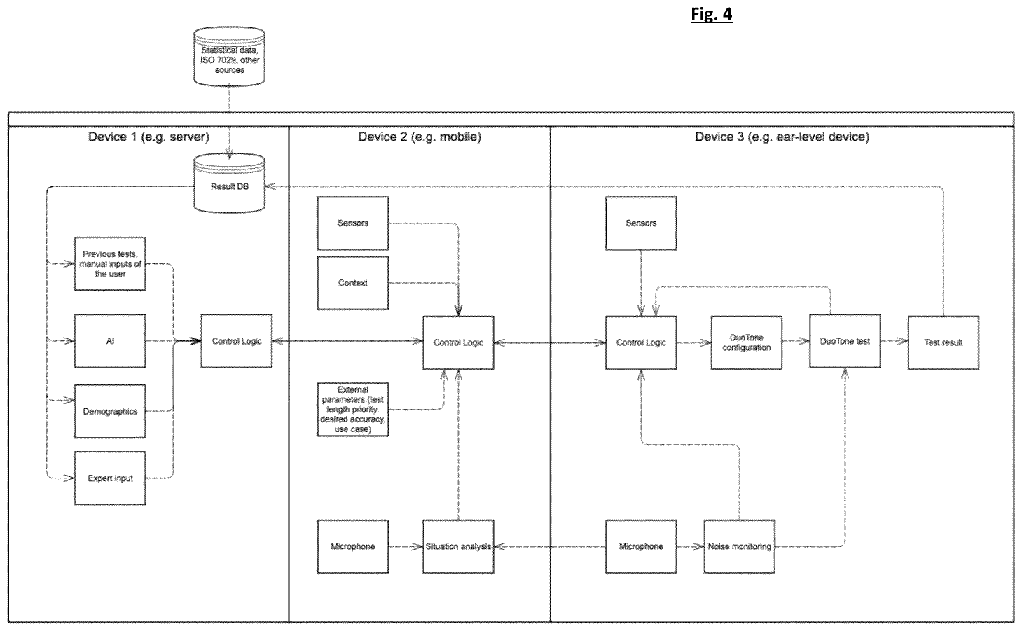

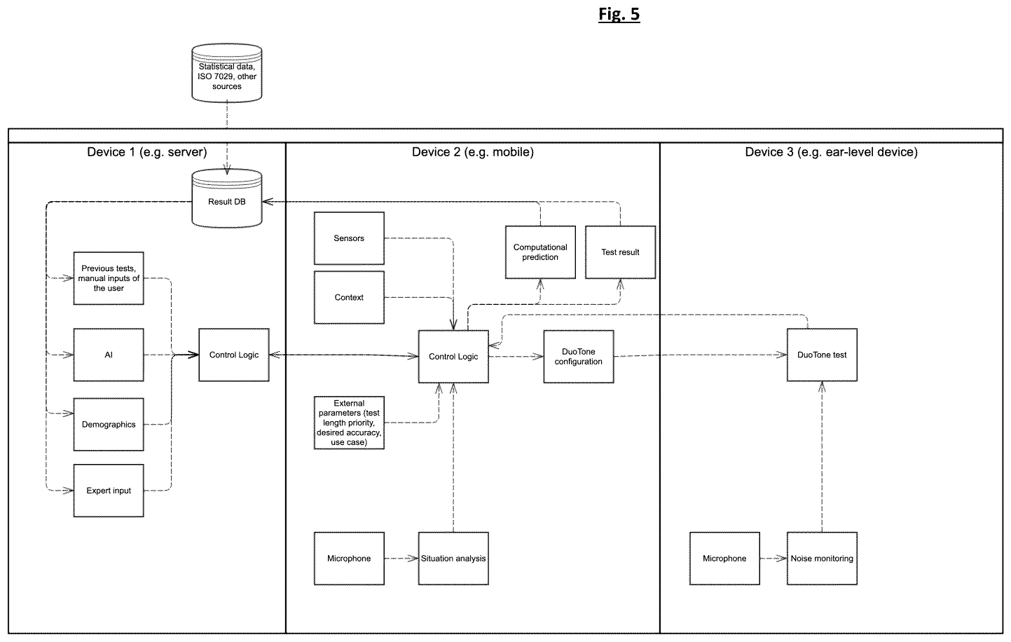

Example implementations of distributed control logic can be found below, see FIG. 3, FIG. 4, and FIG. 5. In FIG. 3 the control logic of the selection module is distributed between two devices, namely a server and a mobile device. Various practical implementations are possible. In one embodiment the prediction is made in the server and the additional inputs to the control logic part in ‘Device 2’ (the mobile device) are used in the determination of the configuration for the hearing assessment procedure. In another embodiment, represented in FIG. 3, the control logic part in the server forwards the collected data to the control logic part in the mobile device, where the prediction of the hearing thresholds is made. The configuration of the test is determined in the mobile device. As shown in FIG. 3, again a DuoTone based approach is adopted. The mobile device is used for generating the tones and for performing the hearing assessment as such. Within the mobile device there is also feedback to the selection module, for conveying not only the user responses obtained during the test, but also the observed noise, so that adaptations to the configuration can be made if necessary. Microphones in the mobile device are used for monitoring the noise conditions.

In FIG. 4 a third device (‘Device 3’) is present compared to FIG. 3. An ear-level device is used when performing the hearing assessment in the example shown in FIG. 4. In the mobile device a partial analysis is performed based on some parameters like e.g. desired accuracy and use case as indicated in the figure and a situation analysis is performed based on inputs from microphones available in the mobile device. The configuration is determined in the ear-level device and provided to the ear-level device, where the tones are generated. Microphones in the ear-level device are used for monitoring the noise conditions. The set-up illustrated in FIG. 4 is an example where the control logic of the selection module is distributed over more than one device. Note also that the test results are stored in a database at the server side.

FIG .5 differs from FIG. 4 in that ‘Device 3’ (e.g., the ear-level device) is only used for carrying out the hearing assessment. The data obtained from the test are fed to the selection module in ‘Device2’ (e.g., a mobile device), more in particular to the control logic part of that module. While performing the test, noise is monitored, thereby making use of a microphone in the ear-level device. The so obtained information is also provided to the selection module.

While the invention has been illustrated and described in detail in the drawings and foregoing description, such illustration and description are to be considered illustrative or exemplary and not restrictive. The foregoing description details certain embodiments of the invention. It will be appreciated, however, that no matter how detailed the foregoing appears in text, the invention may be practiced in many ways. The invention is not limited to the disclosed embodiments.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1.-15. (canceled)

16. A method for performing a hearing assessment procedure comprising:
performing a computational prediction of a hearing loss at at least one frequency different from one or more given frequencies for which a hearing loss indication is obtained by measurement,
selecting a configuration for performing the hearing assessment procedure using at least said predicted hearing loss at said at least one frequency.

17. The method for performing a hearing assessment procedure as in claim 16, wherein said computational prediction is based on at least one of noise analysis, keyword detection in speech, dynamics of hearing loss changes over time.

18. The method for performing a hearing assessment procedure as in claim 16, wherein said computational prediction is based on a volume setting and/or gain setting in a device used to provide test stimuli during the hearing assessment procedure.

19. The method for performing a hearing assessment procedure as in claim 16, wherein said computational prediction is further based on at least one of demographic information, situation analysis, hearing loss indications determined in the past, information on a configuration used in the past, hearing loss observed at non-tested frequencies.

20. The method for performing a hearing assessment procedure as in claim 16, wherein a weighting is applied of two or more pieces of input information when performing said computational prediction.

21. The method for performing a hearing assessment procedure as in claim 16, wherein each stimulus used in said hearing assessment procedure is randomly selected from a set of at least three different testing signals comprising no tone at all, one tone with a first frequency or a plurality of tones with a second frequency different from said first frequency.

22. The method for performing a hearing assessment procedure as in claim 16, wherein said computational prediction of said hearing loss is expressed as a range.

23. The method for performing a hearing assessment procedure as in claim 16, wherein during said hearing assessment procedure additional information for said prediction is supplied and an update of the configuration is determined based on an updated hearing loss prediction.

24. The method for performing a hearing assessment procedure as in claim 16, wherein said hearing assessment procedure is performed both at at least one frequency for which a prediction is computed and at at least one frequency of said one or more given frequencies.

25. The method for performing a hearing assessment procedure as in claim 16, wherein said configuration comprises a starting point for the procedure and a step size to adapt the intensity of stimulus between successive stimuli used in the procedure.

26. The method for performing a hearing assessment procedure as in claim 16, comprising a step of determining a correlation between inputs.

27. A program, executable on a programmable device containing instructions which, when executed, perform the method as in claim 16.

28. A system for performing a hearing assessment, comprising a selection module arranged for receiving parameter values and for selecting a configuration for performing said hearing assessment, processing means for performing a computational prediction of a hearing loss at at least one frequency different from one or more given frequencies for which a hearing loss indication is obtained by measurement, and tone generation means for generating tones according to said selected configuration.

29. The system as in claim 28, wherein said selected module and/or said processing means and/or said tone generation means are integrated.

30. The system as in claim 28, wherein said selected module and/or said processing means are distributed over at least two physically separated devices.
